(19) United States
(12) Patent Application Publication
(10) Pub. No.: US 2026/0183565 A1
(43) Pub. Date: Jul. 2, 2026

(54) PHOTOTHERAPY DEVICE FOR GENITALIA AND PHOTOTHERAPY COVER THEREOF

(71) Applicant: Chung-Yang CHEN, New Taipei City (TW)

(72) Inventor: Chung-Yang CHEN, New Taipei City (TW)

(21) Appl. No.: 19/071,145

(22) Filed: Mar. 5, 2025

(30) Foreign Application Priority Data
Dec. 26, 2024 (TW) 113150943

(51) Int. Cl. A61N 5/06

(52) U.S. Cl. CPC A61N 5/0624; A61N 2005/0626; A61N 2005/0645; A61N 2005/0661

(57) ABSTRACT

A phototherapy device for genitalia includes a charging base and a phototherapy cover. The charging base is equipped with a power supply module that has two power terminals. The phototherapy cover is detachably connected to the charging base and consists of an outer shell, a transparent inner cover, and a circuit module. The transparent inner cover is fixed inside the outer shell, forming a containment space, and it has two charging holes. The inner surface of the transparent inner cover is equipped with multiple light diffusion guide bumps. The circuit module, located in the containment space, includes several light-emitting elements and two charging contacts. These light-emitting elements are aligned with the light diffusion guide bumps, and the two charging contacts are exposed through the two charging holes, enabling an electrical connection with the two power terminals when the phototherapy cover is docked onto the charging base.

This application claims the benefit of Taiwan Patent Application Serial No. 113150943, filed on Dec. 26, 2024, the subject matter of which is incorporated herein by reference.

### BACKGROUND OF THE INVENTION

### (1) Field of the Invention

The invention relates to a phototherapy device, and more particularly to a phototherapy device for genitalia.

### (2) Description of the Prior Art

In recent years, due to the rapid development of science and technology, more and more people are engaged in work that requires long-term use of computers for office work. Therefore, these people are prone to poor blood circulation in the lower body due to long periods of sitting, which in turn leads to various civilization diseases.

As mentioned above, for men, the poor blood circulation caused by sitting for long periods may lead to male infertility due to the reduced ability of the testicles to produce sperm in severe cases.

### SUMMARY OF THE INVENTION

In view of the fact that, in the prior art, office workers who sit for long periods of time are prone to experiencing poor blood circulation. Especially, when blood circulation in the lower body of men is poor, the ability of the testicles to produce sperm is likely to be significantly reduced. Although relevant medical technologies and devices have emerged to address this phenomenon, their designs and efficacy are often limited, making it difficult to effectively provide continuous, gentle, and effective treatment for specific areas. Accordingly, it is a main objective of the present invention to provide an efficient, convenient, and safe genital phototherapy device, specifically designed to address the problem of poor blood circulation in the scrotum area, promotes local blood circulation, improves metabolism and increases the production capacity of testicular sperm. The phototherapy device is paired with a dedicated phototherapy cover to ensure that the light is focused on the scrotum, to prevent scattering and improving treatment efficiency.

On the basis of the above, the necessary technical means adopted by the present invention to address the shortcomings of the prior art are to provide a phototherapy device for genitalia, including a charging base and a phototherapy cover. The charging base is equipped with a power supply module, and the power supply module has two power terminals.

The phototherapy cover is detachably connected to the charging base and comprises an outer shell, a transparent inner cover and a circuit module. The transparent inner cover is fixed inside the outer shell, and forms a shell containment space therebetween, and is provided with two charging holes, and the inner surface of the transparent inner cover is equipped with multiple light diffusion guide bumps.

The circuit module is located in the shell containment space and includes multiple light-emitting elements and two charging contacts. The light-emitting elements are aligned with the light diffusion guide bumps respectively for emitting a light beam with a wavelength between 600 nm and 1200 nm. The two charging contacts are exposed through the two charging holes respectively to enable an electrical connection with the two power terminals when the phototherapy cover is docked onto the charging base.

Among the ancillary technical means derived from the above necessary technical means, the circuit module further includes a control circuit board and a flexible circuit board. The control circuit board has a control switch. The flexible circuit board is electrically connected to the control circuit board, and the light-emitting elements and the two charging contacts are disposed on the flexible circuit board, and the control switch drives the light-emitting elements to emit light when triggered.

Preferably, the phototherapy cover further includes a pressing member. The pressing member is disposed on the outer shell, corresponding to the control switch, and used to trigger the control switch when pressed.

Additionally, the control circuit board further includes an operation indicator light, and the operation indicator light emits light when the control switch is triggered.

Among the ancillary technical means derived from the above necessary technical means, the charging base further includes a base body. The base body is provided with two terminal through holes, the power supply module being disposed inside the base body, and the two power terminals being disposed through the two terminal through holes.

Preferably, the base body is equipped with a power light hole, the power supply module being disposed inside the base body, the power supply module further including a power indicator light, the power indicator light is configured for emitting light toward the power light hole.

Additionally, the base body is further provided with at least one disinfection light hole, the power supply module includes at least one disinfection light, the at least one disinfection light being exposed from at least one disinfection light hole. The base body further includes multiple foot pads.

In summary, the phototherapy device for genitalia of the present invention mainly covers the user's genitalia with the phototherapy cover to perform phototherapy. After the phototherapy cover is used up, it can be placed on the charging base for recharging. In addition, when recharging is required, the phototherapy cover can be placed on the charging base. While the phototherapy cover is charging, the disinfection light arranged on the charging base can also project a disinfection light beam to disinfect and sterilize the inner surface of the phototherapy cover.

The specific embodiments used in the present invention will be further explained through the following embodiments and drawings.

### BRIEF DESCRIPTION OF THE DRAWINGS

The present invention will now be specified with reference to its preferred embodiment illustrated in the drawings, in which:

FIG. 1 is a three-dimensional schematic diagram showing the phototherapy device for genitalia provided by a preferred embodiment of the present invention;

FIG. 2 is a three-dimensional exploded schematic diagram showing the phototherapy device for genitalia provided by a preferred embodiment of the present invention;

FIG. 3 is a three-dimensional exploded schematic diagram showing the phototherapy cover provided by a preferred embodiment of the present invention;

FIG. 4 is a schematic diagram of the A-A section of FIG. 1;

FIG. 5 is a cross-sectional diagram showing that when the phototherapy cover is connected to the charging base for charging, the disinfection light emits light to disinfect the inner side of the phototherapy cover;

FIG. 6 is a system diagram showing the control circuit board of the phototherapy cover of the present invention;

FIG. 7A and FIG. 7B are flowcharts showing the operation of the control circuit board of the present invention;

FIG. 8 is a three-dimensional schematic diagram showing the practical application of the phototherapy cover of the present invention;

FIG. 9 is a cross-sectional view illustrating the practical application of the phototherapy cover of the present invention; and

FIG. 10 is a cross-sectional view showing another practical application of the phototherapy cover of the present invention.

### DESCRIPTION OF THE PREFERRED EMBODIMENT

The present invention disclosed herein is directed to a phototherapy device for genitalia and a phototherapy cover thereof. In the following description, numerous details are set forth in order to provide a thorough understanding of the present invention. It will be appreciated by one skilled in the art that variations of these specific details are possible while still achieving the results of the present invention. In other instances, well-known components are not described in detail in order not to unnecessarily obscure the present invention.

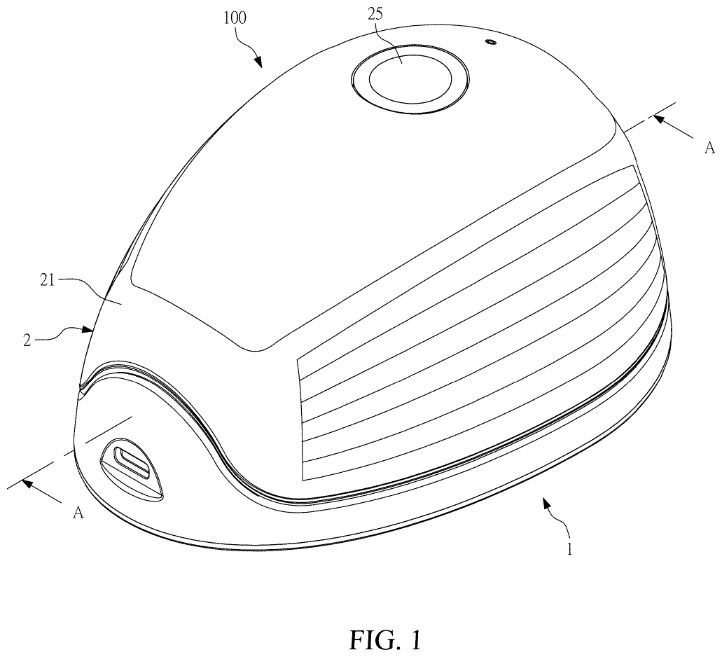

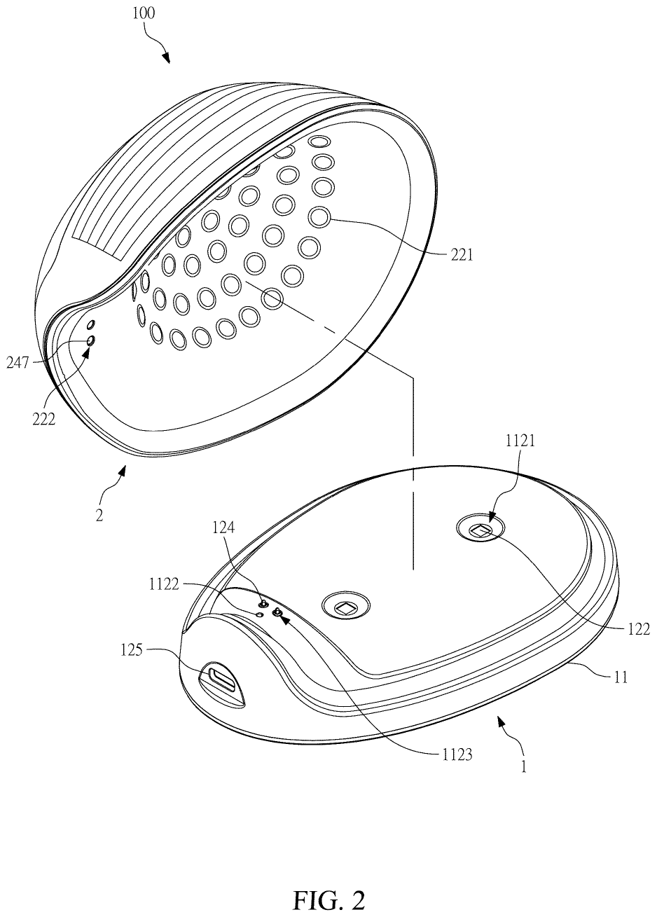

Referring to FIG. 1 and FIG. 2, where FIG. 1 is a three-dimensional schematic diagram illustrating the phototherapy device for genitalia provided by a preferred embodiment of the present invention, and FIG. 2 is a three-dimensional exploded schematic diagram illustrating the phototherapy device for genitalia provided by a preferred embodiment of the present invention.

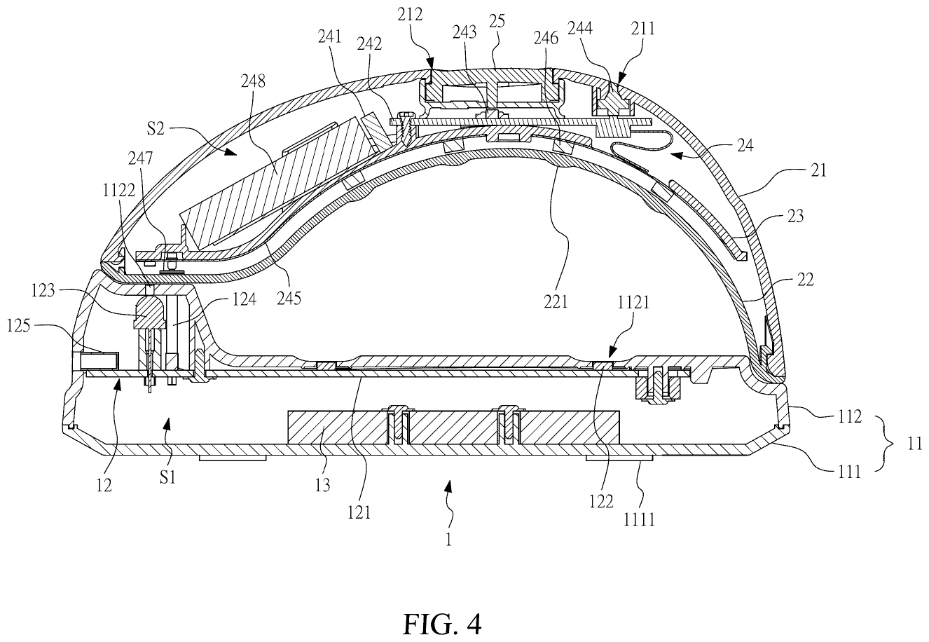

As shown in FIG. 1 and FIG. 2, a phototherapy device for genitalia 100 comprises a charging base 1 and a phototherapy cover 2. The charging base 1 includes a base body 11, a power supply module 12 (as shown in FIG. 4), and a counterweight 13 (as shown in FIG. 4).

The base body 11 includes a base substrate 111 and a base cover 112. The bottom surface of the base substrate 111 is provided with four foot pads 1111 (only one is marked in the figure). The base cover 112 is fixedly connected to the base substrate 111 to enclose a base containment space S1, and the base cover 112 is provided with two disinfection light holes 1121 (only one is marked in the figure), a power light hole 1122 and two terminal through holes 1123 (only one is marked in the figure), wherein the two disinfection light holes 1121, the power light hole 1122 and the two terminal through holes 1123 are respectively connected to the base containment space S1.

The power supply module 12 includes a base circuit board 121, two disinfection lights 122 (only one is marked in the figure), a power indicator light 123, two power terminals 124 (only one is marked in the figure), and a port 125.

The base circuit board 121 is fixed to the base cover 112 in the base containment space S1, wherein the base circuit board 121 can be fixed to the base cover 112 through screws or plastic rivets.

The two disinfection lights 122 are disposed on the base circuit board 121 and exposed in the two disinfection light holes 1121, wherein the disinfection lights 122 are practically disposed on the base circuit board 121 by welding and are correspondingly electrically connected to the circuit of the base circuit board 121. In addition, the disinfection lights 122 of the present embodiment are ultraviolet lights.

The power indicator light 123 is disposed on the base circuit board 121 and exposed from the power light hole 1122, configured for emitting a light beam toward the power light hole 1122.

The two power terminals 124 are disposed on the base circuit board 121 and disposed of through two terminal through holes 1123.

The counterweight 13 is disposed in the base containment space S1 and fixed to the base substrate 111.

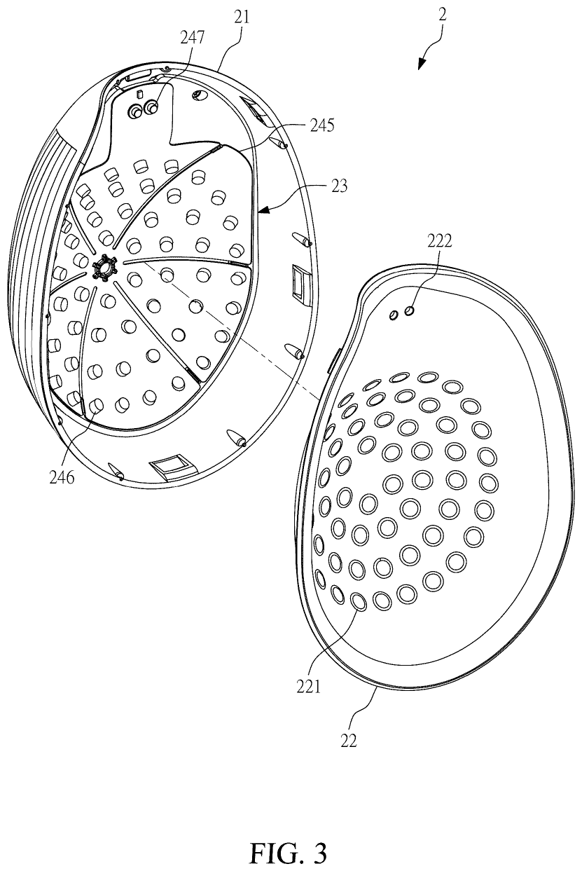

Referring now to FIG. 3 and FIG. 4, where FIG. 3 is a three-dimensional exploded schematic diagram illustrating the phototherapy cover provided by a preferred embodiment of the present invention, and FIG. 4 shows the A-A section of FIG. 1. As shown in FIG. 1 to FIG. 4, the phototherapy cover 2 is detachably connected to the charging base 1 and comprises an outer shell 21, a transparent inner cover 22, a fixed bracket 23, a circuit module 24, and a pressing member 25.

The outer shell 21 is provided with a power light hole 211 and a pressing member setting hole 212. The transparent inner cover 22 is fixed inside the outer shell 21 and forms a shell containment space S2 therebetween. The power light hole 211 and the pressing member setting hole 212 are respectively connected to the shell containment space S2. The inner surface of the transparent inner cover 22 is equipped with multiple light diffusion guide bumps 221 (only one is marked in the figure), and the transparent inner cover 22 is provided with two charging holes 222 (only one is marked in the figure), and the two charging holes 222 are respectively connected to the shell containment space S2. In addition, the light diffusion guide bump 221 of the present embodiment is, for example, an arc-shaped bump with an arc-shaped protruding surface, so as to guide the light beam to be refracted and diffused through the arc-shaped protruding surface. In more detail, the light diffusion guide bump 221 not only guides the light diffusion through the structure but also practically can be doped with scattering particles inside or coated on the surface. The scattering particles can be metal powder or high molecular polymer.

The fixed bracket 23 is disposed inside the shell containment space S2 and fixed to the outer shell 21. The circuit module 24 is located in the shell containment space S2 and includes a temperature sensor 241, a control circuit board 242, a control switch 243, an operation indicator light 244, a flexible circuit board 245, multiple light-emitting elements 246 (only one is marked in the figure), two charging contacts 247 (only one is marked in the figure) and a rechargeable battery 248.

The temperature sensor 241 is disposed in the shell containment space S2 and configured to sense the temperature inside the transparent inner cover 22. In the present embodiment, temperature sensor 241 is, for example, a thermistor temperature sensor such as a negative temperature coefficient (NTC) thermistor or a positive temperature coefficient (PTC) thermistor.

The control circuit board 242 is fixed on one side of the fixed bracket 23. The control switch 243 is disposed on the control circuit board 242, corresponding to the pressing member setting hole 212, and is configured for triggering the control circuit board 242 to operate when pressed. The operation indicator light 244 is disposed on the control circuit board 242, exposed through the power light hole 211, and is configured for emitting light when the control circuit board 242 is operated.

The flexible circuit board 245 is fixed to the opposite side of the fixed bracket 23 relative to the control circuit board 242 and is electrically connected to the control circuit board 242.

The multiple light-emitting elements 246 are disposed on the flexible circuit board 245 and are aligned with the light diffusion guide bumps 221 respectively. When the control switch 243 is pressed, it triggers the control circuit board 242 to drive the light-emitting elements 246 to emit lights. In the present embodiment, the wavelength of the light beam emitted by the light-emitting element 246 is, for example, a red light or near-infrared light beam between 600 nm and 1200 nm, which helps to promote blood circulation or activate cell mitochondria.

The two charging contacts 247 are disposed on the flexible circuit board 245 and correspondingly exposed through the two charging holes 222 respectively to enable an electrical connection with the two power terminals 124 when the phototherapy cover 2 is docked onto the charging base 1. The rechargeable battery 248 is disposed on the fixed bracket 23 and electrically connected to the control circuit board 242 and the flexible circuit board 245.

The pressing member 25 is disposed in the pressing member setting hole 212 of the outer shell 21 and corresponds to the control switch 243. When pressed, the control switch 243 is driven to trigger the control circuit board 242 to operate, thereby driving the light-emitting elements 246 to emit light through the flexible circuit board 245.

As mentioned above, when the phototherapy cover 2 is docked onto the charging base 1, the two power terminals 124 extend into the two charging holes 222 and connect to the two charging contacts 247, so that the base circuit board 121 supplies power to the circuit module 24 to charge the rechargeable battery 248.

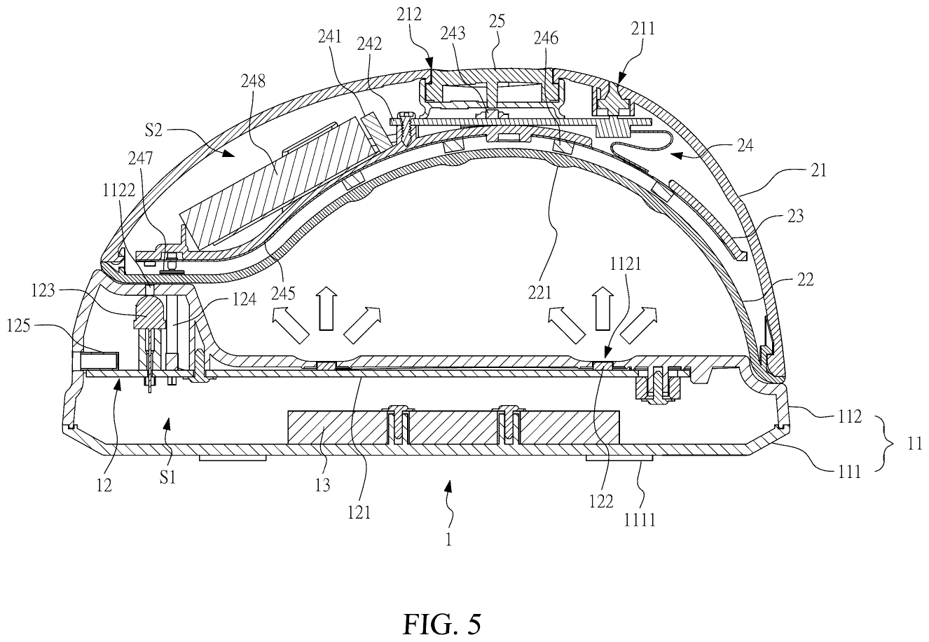

Referring to FIG. 5, where FIG. 5 is a cross-sectional diagram showing that when the phototherapy cover is connected to the charging base for charging, the disinfection light emits light to disinfect the inner side of the phototherapy cover. As shown in FIG. 1 to FIG. 5, when the phototherapy cover 2 is docked onto the charging base 1, not only does the charging base 1 charge the rechargeable battery 248 but the base circuit board 121 also drives the disinfection light 122 to emit light such that it can project a disinfection light beam to disinfect and sterilize the inner surface of the phototherapy cover 2.

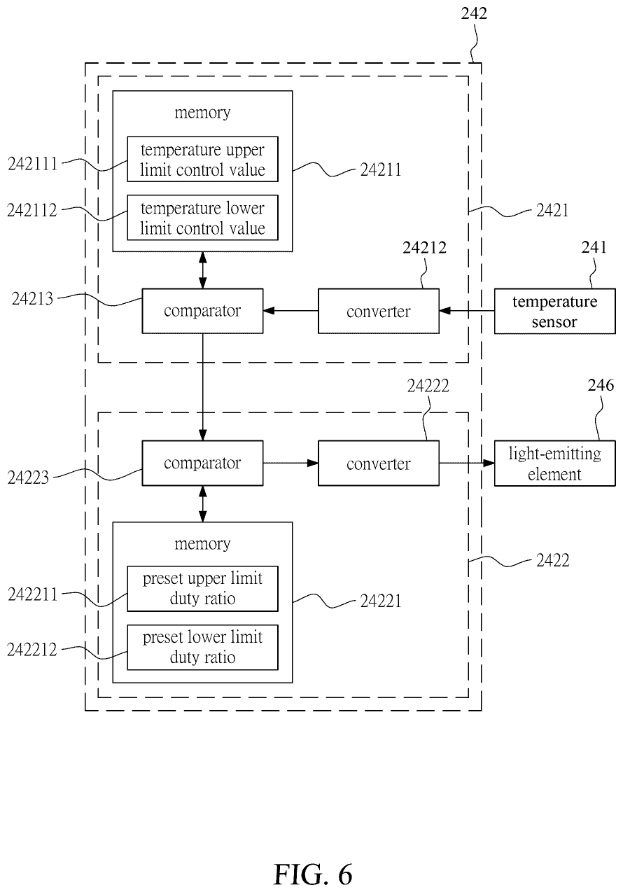

Referring now to FIG. 6, where FIG. 6 is a system diagram showing the control circuit board of the phototherapy cover of the present invention. As shown in FIG. 1 to FIG. 6, the control circuit board 242 practically further includes a temperature determination unit 2421 and a duty ratio determination control unit 2422. The temperature determination unit 2421 includes a memory 24211, a converter 24212, and a comparator 24213. The memory 24211 includes a temperature upper limit control value of 242111 and a temperature lower limit control value of 242112 built-in. The converter 24212 is electrically connected to the temperature sensor 241. In the present embodiment, the converter 24212 is used to convert the resistance signal transmitted by the temperature sensor 241 into a voltage signal. The comparator 24213 is electrically connected to the memory 24211 and the converter 24212, and used to compare the temperature value represented by the voltage signal transmitted by the converter 24212 with the temperature upper limit control value 242111 or the temperature lower limit control value 242112.

The duty ratio determination control unit 2422 includes a memory 24221, a converter 24222, and a comparator 24223. The memory 24221 includes a preset upper limit duty ratio of 242211 and a preset lower limit duty ratio of 242212. The converter 24222 is electrically connected to the light-emitting elements 246, and the comparator 24223 is electrically connected to the comparator 24213, the memory 24221 and the converter 24222 of the temperature determination unit 2421. Wherein, the comparator 24223 further compares the current duty ratio with the preset upper limit duty ratio 242211 or the preset lower limit duty ratio 242212 according to the comparison result of the comparator 24213, and controls the converter 24222 to output a driving signal to the light-emitting elements 246 accordingly, thereby driving the light-emitting elements 246 to emit light.

It should be noted that, in the present embodiment, the comparators 24213 and 24223 may be firmware with an operating program, and the temperature determination unit 2421 and the duty ratio determination control unit 2422 may be, for example, logic circuits integrated into a chip module.

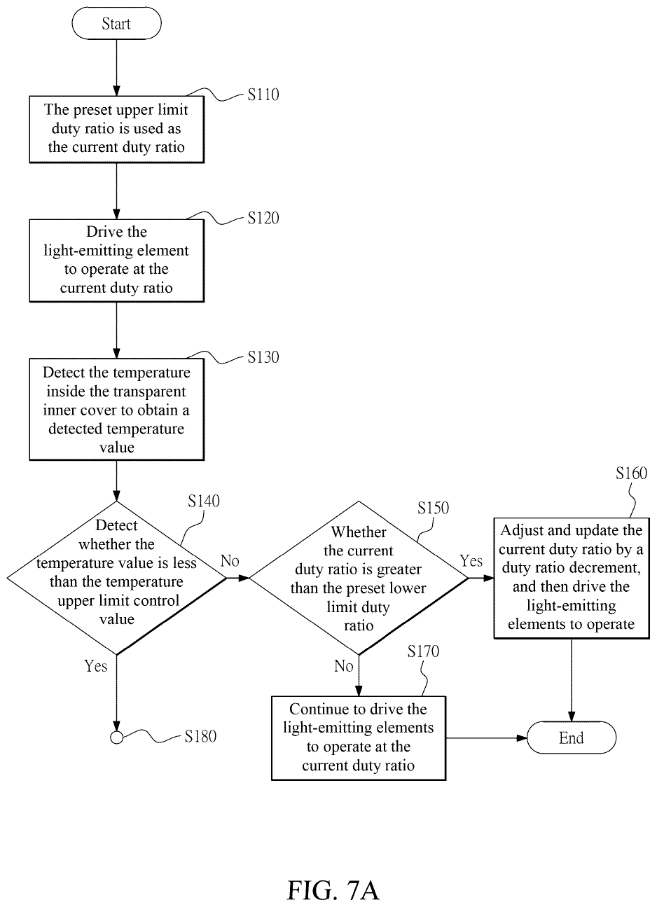

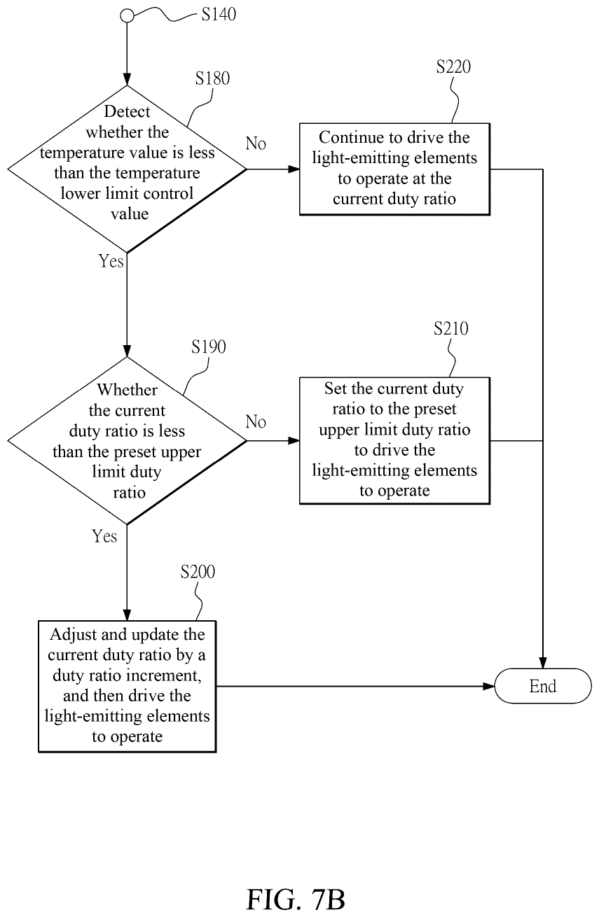

Referring now to FIG. 7A and FIG. 7B, where FIG. 7A and FIG. 7B are flowcharts showing the operation of the control circuit board of the present invention. As shown in FIG. 4 to FIG. 7B, when the control circuit board 242 of the present embodiment is triggered by the control switch 243, the duty ratio determination control unit 2422 first performs step S110 to set the preset upper limit duty ratio 242211 as the current duty ratio and then proceeds to step S120, the duty ratio determination control unit 2422 then drives the light-emitting elements 246 to operate at the current duty ratio.

After step S120, step S130 is to utilize the temperature sensor 241 to detect the temperature inside the transparent inner cover 22 to obtain a detected temperature value. After the detected temperature value inside the transparent inner cover 22 is obtained in step S130, step S140 uses the temperature determination unit 2421 to compare and determine whether the detected temperature value is less than the temperature upper limit control value 242111.

Next, when step S140 determines that the detected temperature value is not less than the temperature upper limit control value 242111, but is greater than or equal to the temperature upper limit control value 242111, step S150 will be performed. In step S150, the duty ratio determination control unit 2422 is used to compare and determine whether the current duty ratio is greater than a preset lower limit duty ratio 242212.

As mentioned above, if step S150 determines that the current duty ratio is greater than the preset lower limit duty ratio 242212, step S160 will be performed. Step S160 is that the duty ratio determination control unit 2422 adjusts and updates the current duty ratio by a duty ratio decrement and then drives the light-emitting elements 246 to operate. However, if step S150 determines that the current duty ratio is less than or equal to the preset lower limit duty ratio 242212, step S170 will then be performed. In step S170, the duty ratio determination control unit 2422 continues to drive the light-emitting elements 246 to operate at the current duty ratio.

On the other hand, when step S140 determines that the detected temperature value is less than the temperature upper limit control value 242111, step S180 is then performed. Step S180 is to use the temperature determination unit 2421 to compare and determine whether the detected temperature value is less than the temperature lower limit control value 242112. At this time, if step S180 determines that the detected temperature value is less than the temperature lower limit control value 242112, step S190 will continue to be performed. Step S190 will further compare and determine whether the current duty ratio is less than the preset upper limit duty ratio 242211. At this time, if step S190 determines that the current duty ratio is less than the preset upper limit duty ratio 242211, step S200 will continue to be performed, otherwise step S210 will continue to be performed.

As mentioned above, step S200 is that the duty ratio determination control unit 2422 adjusts and updates the current duty ratio by a duty ratio increment, then drives the light-emitting elements 246 to operate. In step S210, the duty ratio determination control unit 2422 sets the current duty ratio to the preset upper limit duty ratio 242211 to drive the light-emitting elements 246 to operate.

Besides, when the detected temperature value is determined to be greater than or equal to the temperature lower limit control value 242112 in step S180, step S220 is then performed. In step S220, the duty ratio determination control unit 2422 continues to drive the light-emitting elements 246 to operate at the current duty ratio.

For example, the temperature upper limit control value 242111 of the present embodiment is 37° C., the temperature lower limit control value 242112 is 34° C., the preset upper limit duty ratio 242211 is 50:50, the preset lower limit duty ratio 242212 is 10:90, and the duty ratio increment and duty ratio decrement are, for example, 10%. Under this condition, the control circuit board 242 will first use 50:50 as the current duty ratio to drive the light-emitting elements 246 to emit lights, and then when the temperature inside the transparent inner cover 22 is higher than 37° C. (step S140), it is compared and determined that 50:50 is greater than 10:90 (step S150), so the current duty ratio is reduced (step S160). When the duty ratio is less than or equal to 10:90, the operation will continue with the current duty ratio as in step S170.

As mentioned above, when the temperature inside the transparent inner cover 22 is less than 37° C. (step S140), a further comparison is made to see whether the temperature is less than the temperature lower limit control value 242112 (step S180). If the detected temperature value is less than the temperature lower limit control value 242112, a further comparison will be made to see whether the current duty ratio is less than the preset value 50:50 (step S190). However, when the current duty ratio is less than 50:50, the temperature will be increased to accelerate the temperature rise (step S200). Otherwise, the current duty ratio will be set to the preset upper limit duty ratio 242211 (50:50) to continue the operation (step S210).

Besides, when the temperature inside the transparent inner cover 22 is less than 37° C. but greater than or equal to the temperature lower limit control value 242112, the operation will continue at the current duty ratio (step S220).

It should be noted that the above duty ratio of 10:90 refers to the light flashing frequency of 10% of the light time and 90% of the stop light time, while 50:50 refers to the light flashing frequency of 50% of the light time and 50% of the stop light time. Therefore, the lower the duty ratio, the slower the temperature rising rate, and the higher the duty ratio, the faster the temperature rising rate.

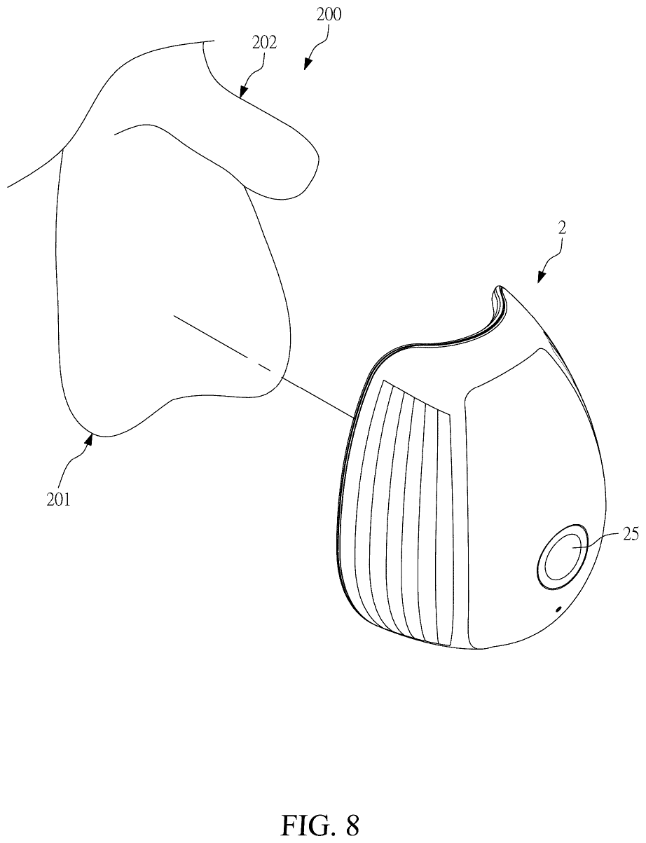

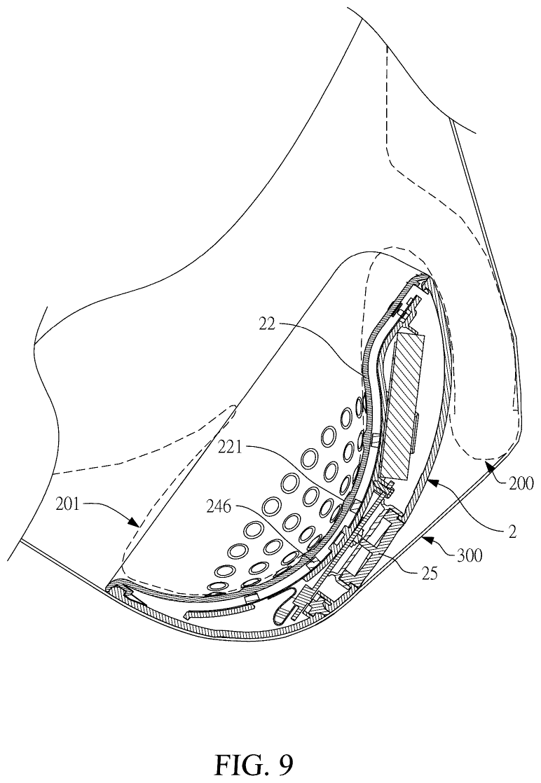

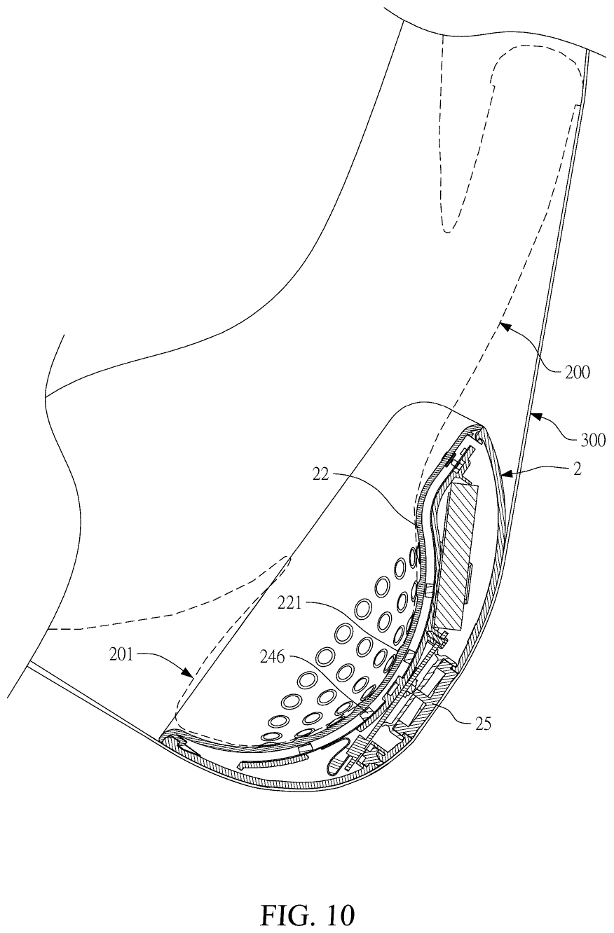

Referring now to FIG. 8 to FIG. 10, where FIG. 8 is a three-dimensional schematic diagram showing the practical application of the phototherapy cover of the present invention, FIG. 9 is a cross-sectional view showing the practical application of the phototherapy cover of the present invention, and FIG. 10 is a cross-sectional view showing another practical application of the phototherapy cover of the present invention.

As shown in FIG. 1 to FIG. 10, in the practical application, the phototherapy cover 2 of the present invention is used to cover a scrotum 201 of a genitalia 200, and the phototherapy cover 2 can be secured by wearing underwear 300, and the penis 202 can be placed between the phototherapy cover 2 and the underwear 300.

As mentioned above, the user can activate the light-emitting elements 246 by pressing the pressing member 25, whereby when the phototherapy cover 2 is placed on the scrotum 201, the light beam generated by the light-emitting elements 246 will be directly projected onto the transparent inner cover 22 and will be evenly scattered to the scrotum 201 through the corresponding light diffusion guide bumps 221, thereby promoting blood circulation and metabolism in the scrotum 201 and internal testicles. Besides, since the light diffusion guide bumps 221 make the inner surface of the transparent inner cover 22 uneven, a gap can be created between the scrotum 201 and the transparent inner cover 22 to prevent excessive closeness and discomfort.

In summary, since men who sit in the office for a long period are prone to poor blood circulation in the lower body, which leads to a decrease in the ability of the testicles to produce sperm, the phototherapy device for genitalia of the present invention mainly uses a phototherapy cover for the user to place in his underwear to cover the scrotum, to provide phototherapy to the scrotum at a close distance. It can indeed effectively improve the blood circulation problems of the genitalia.

While the present invention has been particularly shown and described with reference to a preferred embodiment, it will be understood by those skilled in the art that various changes in form and detail may be made without departing from the spirit and scope of the present invention.

What is claimed is:

1. A phototherapy device for genitalia, comprising:
a charging base, equipped with a power supply module, the power supply module having two power terminals; and
a phototherapy cover, detachably connected to the charging base, comprising:
an outer shell;
a transparent inner cover, fixed inside the outer shell, forming a shell containment space therebetween, having two charging holes, the inner surface of the transparent inner cover being equipped with multiple light diffusion guide bumps; and
a circuit module, located in the shell containment space, including multiple light-emitting elements and two charging contacts, the light-emitting elements being aligned with the light diffusion guide bumps respectively for emitting a light beam with wavelength between 600 nm and 1200 nm, the two charging contacts being exposed through the two charging holes respectively for enabling an electrical connection with the two power terminals when the phototherapy cover is docked onto the charging base.

2. The phototherapy device for genitalia of claim 1, wherein the circuit module further includes:
a control circuit board, having a control switch; and
a flexible circuit board, electrically connected with the control circuit board, the light-emitting elements, and the two charging contacts being disposed on the flexible circuit board, the control switch driving the light-emitting elements to emit light when triggered.

3. The phototherapy device for genitalia of claim 2, wherein the phototherapy cover further includes a pressing member, the pressing member being disposed on the outer shell, corresponding to the control switch and used to trigger the control switch when pressed.

4. The phototherapy device for genitalia of claim 2, wherein the control circuit board further includes an operation indicator light, the operation indicator light emitting light when the control switch is triggered.

5. The phototherapy device for genitalia of claim 1, wherein the charging base further includes a base body, the base body being provided with two terminal through holes, the power supply module being disposed inside the base body, the two power terminals being disposed through the two terminal through holes.

6. The phototherapy device for genitalia of claim 5, wherein the base body is further provided with at least one disinfection light hole, the power supply module further including at least one disinfection light, the at least one disinfection light being exposed from the at least one disinfection light hole.

7. A phototherapy cover, detachably connected to a charging base, comprising:
an outer shell;
a transparent inner cover, fixed inside the outer shell, forming a shell containment space therebetween, having two charging holes, the inner surface of the transparent inner cover being equipped with multiple light diffusion guide bumps; and
a circuit module, located in the shell containment space, including multiple light-emitting elements and two charging contacts, the light-emitting elements being aligned with the light diffusion guide bumps respectively, the two charging contacts being exposed through the two charging holes respectively for enabling an electrical connection with two power terminals of the charging base when the phototherapy cover is docked onto the charging base.

8. The phototherapy cover of claim 7, wherein the circuit module further includes:
a control circuit board, having a control switch; and
a flexible circuit board, electrically connected with the control circuit board, the light-emitting elements, and the two charging contacts being disposed on the flexible circuit board, the control switch driving the light-emitting elements to emit light when triggered.

9. The phototherapy cover of claim 8, wherein the circuit module further includes a temperature sensor disposed inside the shell containment space and electrically connected to the control circuit board for sensing the temperature of the shell containment space, the control circuit board further including:
a temperature determination unit, having a temperature upper limit control value and a temperature lower limit control value built-in, configured for comparing the temperature of the shell containment space with the temperature upper limit control value or the temperature lower limit control value, wherein the temperature upper limit control value is 37° C. and the temperature lower limit control value is 34° C.; and
a duty ratio determination control unit, having a preset upper limit duty ratio and a preset lower limit duty ratio built-in, configured for further comparing a current duty ratio with the preset upper limit duty ratio or a preset lower limit duty ratio according to the temperature comparison result of the temperature determination unit, the light-emitting elements being controlled to emit light accordingly.

10. The phototherapy cover of claim 8, wherein the phototherapy cover further includes a pressing member, the pressing member being disposed on the outer shell, corresponding to the control switch and used to trigger the control switch when pressed.
